(19) United States
(12) Patent Application Publication
(10) Pub. No.: US 2006/0188952 A1
(43) Pub. Date: Aug. 24, 2006

(54) Homogeneous fluorescence assay for kinases, phosphatases, and phosphodiesterases

(75) Inventor: Franz-Josef Meyer-Almes, Iserlohn (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/525,601

(22) PCT Filed: Aug. 18, 2003
(86) PCT No.: PCT/EP03/09121

§ 371 Date: Mar. 13, 2006

(30) Foreign Application Priority Data
Aug. 26, 2002 (DE) 102 39 005.3

(51) Int. Cl. C12Q 1/48; C12Q 1/34

(52) U.S. Cl. 435/15; 435/18

(57) ABSTRACT

The present invention relates to a homogeneous assay method of quantitative measurement of kinase, phosphatase and phosphodiesterase (PDE) reactions. The method may be used both in a direct and in a competitive assay format.

The present invention relates to a homogeneous assay method of quantitative measurement of kinase, phosphatase and phosphodiesterase (PDE) reactions. The method may be used both in a direct and in a competitive assay format.

Protein (de)phosphorylation is a general regulatory mechanism which is used by cells to selectively modify proteins which impart regulatory signals from outside to the nucleus. The proteins which carry out these biochemical modifications belong to the group of the kinases and phosphatases, respectively. Phosphodiesterases hydrolyse the secondary messenger cAMP or cGMP and, in this way, likewise influence cellular signal transduction pathways. Therefore, these enzymes are useful and highly interesting target molecules of pharmaceutical and plant protection research.

Traditional methods of measuring the state of phosphorylation of cellular proteins are based on incorporation of radioactive ³²P-orthophosphate. The ³²P-phosphorylated proteins are separated on a gel and subsequently visualized using a phospho-imager. Alternatively, phosphorylated tyrosine residues may be bound via binding of radiolabelled anti-phosphotyrosine antibodies and detected by immunoassays, for example immunoprecipitation or blotting. Since these methods need to detect radioisotopes, they are time-consuming and also, owing to the safety aspects involved in the handling of radioactive substances, not suitable for ultra high throughput screening (uHTS).

In more recent methods, the radioactive immunoassays are replaced with ELISAs (enzyme-linked immunosorbent assays). These methods use purified substrate proteins or synthetic peptide substrates which have been immobilized to a substrate surface. After a kinase action, the extent of phosphorylation is quantified by the binding of anti-phosphotyrosine antibodies coupled to an enhancer enzyme such as peroxidases, for example, to the phosphorylated immobilized substrates.

Epps. et al. (U.S. Pat. No. 6,203,994) describe a fluorescence-based HTS assay for protein kinases and phosphatases, which makes use of fluorescently labelled phosphorylated reporter molecules and antibodies which bind specifically to the phosphorylated reporter molecules. Binding is measured by means of fluorescence polarization, fluorescence quenching or fluorescence correlation spectroscopy (FCS). This method has the intrinsic disadvantage of only good generic antibodies (e.g. clone PT66, PY20, Sigma) being available for phosphotyrosine substrates. Only a few examples of suitable anti-phosphoserine or anti-threonine antibodies have been reported (e.g. Bader B. et al., Journal of Biomolecular Screening, 6, 255 (2001), Panvera kit no. P2886). These antibodies, however, have the property of recognizing not only phosphoserine but also the neighbouring amino acids as epitope. It is known, however, that kinases function very substrate-specifically and that substrate sequences can differ greatly. Therefore, anti-phosphoserine antibodies cannot be used as generic reagents.

Perkin Elmer (Wallac) provide an assay for tyrosine kinases, which is based on time-resolved fluorescence and an energy transfer from europium chelates to allophycocyanine (see also EP 929 810). Here too, the process is limited essentially to tyrosine kinases, due to the use of antibodies.

Recently, Molecular Devices have provided nanoparticles with charged metal cations oh the surface as a generic binding reagent suitable for phosphorylation reactions on tyrosine as well as and on serine and threonine. However, the binding reaction is carried out at a strongly acidic pH of approx. 5 and a high ionic strength. Binding of the nanoparticles therefore requires a step of greatly diluting the reaction into the target buffer, which is problematic with a total assay volume of 10 μl in the 1536 format in uHTS. Binding is measured here, too, by means of fluorescence polarization.

Finally, Nikorov introduced polyionic polymers as binding reagents of phosphorylation reactions. He described poly-amino acids such as, for example, poly-histidine, poly-L-lysine and poly-L-arginine (U.S. Pat. No. 6,287,774, Nikorov et al. Anal. Biochm. 278, 206-212 (2000)). However, Nikorov's invention refers exclusively to fluorescence polarization as the method of measurement, which is relatively complicated and currently does not yet allow parallel measurement of a microtitre plate (MTP). Therefore, measurement times for a 1536-MTP would be very high and measuring enzyme kinetics in parallel would not be possible. Moreover, fluorescence polarization, as a method, is limited to very small fluorescent substrates. In addition, polyethylenimines have not been mentioned explicitly as binding reagents.

The present invention breaks down the limitation of U.S. Pat. No. 6,203,994 of being restricted to (de)phosphorylation reactions on tyrosine by using polycationic polymers rather than antibodies. This enables any kinase and phosphatase reactions on serine, threonine and tyrosine and also phosphodiesterase reactions to be measured. While Nikorov (U.S. Pat. No. 6,287,774) is unable to measure a microtitre plate with 96, 384 or even 1536 samples in parallel by means of fluorescence polarization, according to the current state of the art, my invention, owing to the simple measurement technique, discloses parallel measurement even of enzyme kinetics with high time resolution. In addition, measurements of fluorescence intensity, compared to those of fluorescence polarization, make greater sensitivity with shorter measurement times possible.

Another advantage, compared to Nikorov, is the possibility of using polyethylenimine which is substantially cheaper and more stable to hydrolysis.

### DESCRIPTION OF THE INVENTION

The present invention employs polycationic polymers having fluorescence-quenching properties, in order to measure kinase, phosphatase and phosphodiesterase reactions. The assay method does not include any washing steps and is also perfectly suitable for miniaturized assays in total volumes of 10 μl and less. The polyionic polymer which is a useful universal and generic binding reagent for molecules having at least one singly bonded phosphate group may be employed unmodified or labelled with quencher dyes such as, for example, Dabcyl or QSY35.

The direct assay format essentially comprises the following steps:
i) A fluorescent reactant is converted by a kinase, phosphatase or PDE reaction into a fluorescent product which differs from the reactant by at least one singly bonded phosphate group.
ii) A polycationic polymer containing quencher groups is added (prior to, during or after the reaction) and binds either the phosphorylated fluorescent reactant or the phosphorylated fluorescent product, with the fluorescence of the phosphorylated reactant or the phosphorylated product being quenched by the quencher groups on the polycationic polymer.
iii) The reaction turnover is quantified by measuring the fluorescence intensity and/or fluorescence lifetime. The reaction kinetics may be followed if the polycationic polymer is added prior to or during the reaction.

The competitive assay format comprises the following steps:
i) A non-fluorescent reactant is converted by a kinase, phosphatase or PDE reaction into a non-fluorescent product which differs from the reactant by at least one singly bonded phosphate group.
ii) A polycationic polymer containing quencher groups and a fluorescent, phosphorylated reporter reagent are added (prior to, during or after the reaction). A complex of the polycationic polymer and the reporter reagent forms if no phosphorylated reactant or phosphorylated product is present, with the fluorescence of the phosphorylated reactant or the phosphorylated product being quenched by the quencher groups on the polycationic polymer. The phosphorylated reactant or phosphorylated product, when present, competes with the reporter reagent for binding to the polycationic polymer, thereby stopping fluorescence quenching of the reporter reagent.
iii) The reaction turnover is quantified by measuring the fluorescence intensity and/or fluorescence lifetime. The reaction kinetics may be followed if the polycationic polymer and the fluorescent reporter reagent are added prior to or during the reaction.

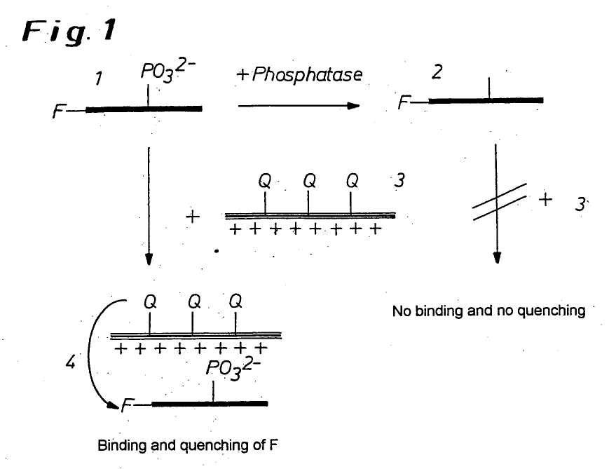

Phosphatase assays are configured in such a way that a fluorescent phosphorylated substrate peptide or substrate protein (1) is first dephosphorylated by a phosphatase. After the reaction, the polyionic polymer (3) is added. If the enzyme is active, then the polyionic polymer does not bind to the fluorescent dephosphorylated substrate (2) and the unquenched fluorescence is high. If the enzyme is inactive or inhibited, then the polyionic polymer binds to the fluorescent phosphorylated substrate and quenches the fluorescence of the latter (complex, 4) (see FIG. 1).

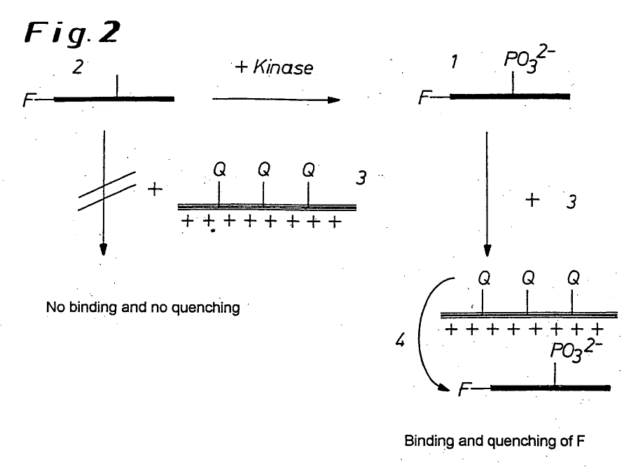

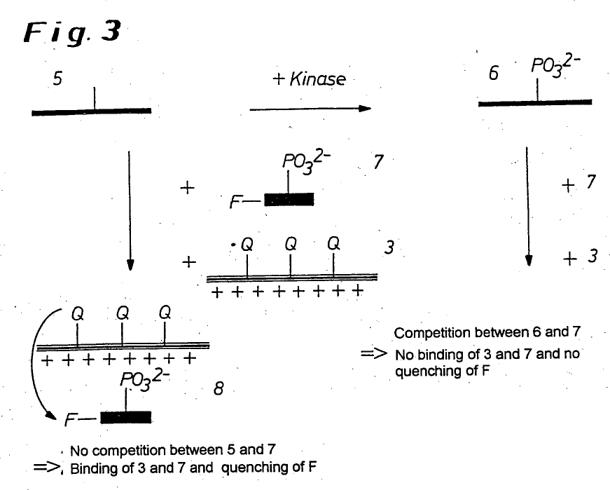

Kinase assays may be designed either directly or competitively. The direct kinase assay employs a non-phosphorylated fluorescent substrate peptide or substrate protein which contains at least one serine or threonine or tyrosine. The polyionic polymer may be added immediately at the start of the reaction or else after the reaction. If the kinase is active, then the substrate is phosphorylated and bound by the polyionic polymer, with the substrate fluorescence being quenched. If the kinase is inhibited or inactive, then the substrate fluorescence remains high (see FIG. 2). A competitive kinase assay employs, besides a non-fluorescent substrate peptide or substrate protein (5), a fluorescent phosphorylated peptide or protein (reporter reagent, 7) which is bound by the polyionic polymer, with the fluorescence of the reporter reagent being quenched. The polymer may be added at the start of or after the enzyme reaction. The substrate competes increasingly with the reporter reagent for binding to the polyionic polymer (complex, 8), to the extent to which the substrate is phosphorylated (6). Consequently, less and less reporter reagent is bound by the polyionic polymer and quenching of the fluorescence of the reporter gene decreases. (see FIG. 3).

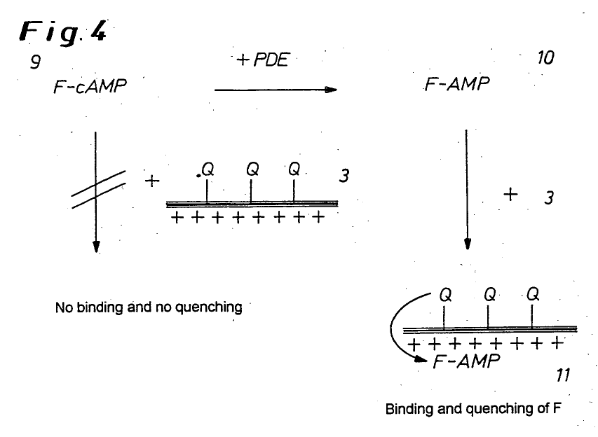

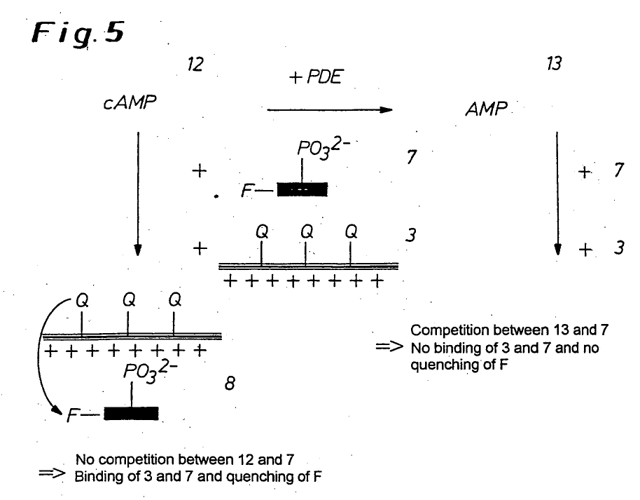

Similarly to kinase assays, Phosphodiesterase assays may also be configured directly or competitively. In the direct mode, a fluorescent cAMP or cGMP derivative (9) is used which is not bound by the polyionic polymer. In this case, no fluorescence quenching takes place. As soon as the cyclic nucleotide derivatives are hydrolysed and thus a singly bonded phosphate group is formed, the polyionic polymer binds the fluorescent nucleotide monophosphate formed (10) and quenches the fluorescence thereof (complex, 11) (see FIG. 4). Analogously to the competitive kinase assay, the competitive mode employs a fluorescent phosphorylated reporter reagent (7) and non-fluorescent cAMP or cGMP derivatives (12). If the phosphodiesterase is active, then nucleotide monophosphate (13) is formed which competes with the reporter reagent for binding to the polyionic polymer, i.e. the fluorescence of the reporter reagent is no longer quenched. If the enzyme is inhibited or inactive, then the polyionic polymer binds to the reporter reagent and quenches the fluorescence thereof (see FIG. 5).

The measurement principle in all of the assay variants presented is based on quenching the fluorescence of a substrate or a reporter reagent. The fluorescence-quenching mechanism may be, for example, a Förster energy transfer to a non-fluorescent dye. This also influences the fluorescence lifetime so that it is possible to measure binding of the polyionic polymer to the fluorescent substrate or reporter reagent by means of measuring the fluorescence lifetime. A change in fluorescence lifetime may also be measured when the fluorophore F is sufficiently close to the phosphorylated amino acid to which the polyionic polymer binds. In this case, the polymer does not need to be labelled with quencher dyes.

The present invention also relates to a homogeneous assay method for kinases, phosphatases and phosphodiesterases by direct distinction of reactants and products of the kinase, phosphatase and phosphodiesterase reactions, comprising the following steps:
a) the substrate of kinase reactions consists of a fluorescent peptide or a fluorescent protein which must contain at least one serine or at least one threonine or at least one tyrosine which is phosphorylatable by the kinase.
The substrate of phosphatase reactions consists of a fluorescent peptide or a fluorescent protein which must contain at least one phosphorylated serine or at least one phosphorylated threonine or at least one phosphorylated tyrosine which is dephosphorylatable by the phosphatase.
The substrate of a phosphodiesterase is a fluorescent cAMP or cGMP derivative which is converted by the phosphodiesterase into the corresponding AMP or GMP derivative with a free phosphate group.
b) The addition of unmodified or quencher dye-modified polycationic polymers selectively binding the reactant or the product of the enzyme reaction, which contains at least one singly bonded phosphate group, and in the process quenching the fluorescence of the bound reactant or product distinguishes between the reactant and product of kinase, phosphatase and phosphodiesterase reactions.
c) Bindung of the polycationic polymers can be detected by fluorescence measurements.

Alternatively, the kinase and phosphodiesterase assays may also be configured competitively:

The substrate of kinase reactions consists of a non-fluorescent peptide or a non-fluorescent protein which must contain at least one serine or at least one threonine or at least one tyrosine which is phosphorylatable by the kinase.

The substrate of a phosphodiesterase is a non-fluorescent cAMP or cGMP derivative which is converted by the phosphodiesterase into the corresponding AMP or GMP derivative with a free phosphate group.

Additionally, one at least monophosphorylated fluorescent peptide or protein (reporter reagent) to which the polycationic polymer binds, is added.

The addition of unmodified or quencher dye-modified polycationic polymers results in binding of the receptor reagent and quenching of the fluorescence thereof. To the extent to which the product which contains at least one singly bonded phosphate group is formed by the kinase or phosphodiesterase reaction, this phosphorylated product competes with the reporter reagent for binding to the polycationic polymer. This stops the reporter reagent from binding to the polycationic polymer, and the fluorescence of the reporter reagent increases again to the unquenched value.

Binding of the polycationic polymers can be detected by fluorescence measurements.

### EXAMPLES

1) Preparation of the Conjugate of Dabcyl and Polyethylenimine (PEI880-Dabcyl):

Material: Polyethylenimine (PEI880), Fluka no. 03880, MW approx. 600-1000 kDa
4-((4-(Dimethylamino)phenyl)azo)benzoic acid, succinimidyl ester (Dabcyl-SE),
Molecular Probes, no. D-2245
NaHCO₃, Na₂CO₃, Sigma, nos. 6329, 6392
Hydroxylamine hydrochloride,
10 M NaOH
Dimethyl sulphoxide, Sigma-Aldrich, no. 41640
Sephadex G-25 NAP-10 columns, Pharmacia
Procedure: Carbonate buffer pH 9.0, 1M:
10 ml of 1M NaHCO₃
+2.5 ml of 1M Na₂CO₃
Coupling of Dabcyl to PEI880:
100 μl of 0.15 g/ml PEI880, pH 7.0
+100 μl of 1M carbonate buffer pH 9.0
+600 μl of H₂O
+200 μl of Dabcyl-SE-Lösung, 10 mg/ml in DMSO (freshly dissolved)
The reaction mix is incubated with gentle shaking at room temperature in the dark for 1 hour. The reaction is then stopped by adding 100 μl of 1.5 M hydroxylamine pH 8.5 and incubating at room temperature in the dark for 1 hour. Subsequently, the PEI880-Dabcyl conjugate is removed from excess Dabcyl dye by gel filtration on an NAP-10 column.
Result: The first orange band of the chromatographic purification represented the product, Dabcyl-labelled PEI880. Excess and deactivated . . . ???-Dabcyl dye which is likewise orange remained on the column.
   2) Influence of Ionic Strength on Binding of a Phosphoserine Peptide to PEI880-Dabcyl
Material: Fluorescein-(GRPRTpSSFAEG) (Tracer), Panvera, kit no. P2886 PEI880-Dabcyl conjugate
Procedure: 10 nM tracer and 0.6 μM PEI880-Dabcyl conjugate were each incubated in HEPES buffer pH 7.5 in the presence of 0, 150 mM and 1 M NaCl in each case at room temperature in the dark for at least 20 minutes. The reaction mixtures were pipetted on 1536-microtitre plates, using an Igel pipetter (Cybio). Finally, fluorescence intensity and fluorescence polarization were measured simultaneously in a Tecan Ultra.
Result: The tracer fluorescence was quenched in the absence of NaCl, with no quenching taking place at relatively high ionic strength. Fluorescence polarization measurements showed no increase only at 150 mM and 1 M NaCl, confirming at the same time that no binding between tracer and polymer took place under the said conditions. This is evidence of the ionic character of the bond.
   3) Binding of a Fluorescent Phosphotyrosine Peptide to PEI880-Dabcyl:
Material: Fluorescein-C6-TEGQpYQPQP (F1-P1), synthesis by Eurogentec PEI880-Dabcyl conjugate
Procedure: 10 nM F1-P1 were each incubated in the presence of 150 mM NaCl in 50 mM HEPES buffer pH 7.5 at PEI880-Dabcyl concentrations of from 10 pM to 120 μM in each case at room temperature in the dark for at least 20 minutes. The reaction mixtures were pipetted on 1536-microtitre plates, using an Igel pipetter (Cybio). Finally, fluorescence intensity and fluorescence polarization were measured simultaneously in a Tecan Ultra.
Result: The phosphotyrosine peptide F1-P1 is also bound by PEI880-Dabcyl. As in the case of the phosphoserine peptide (Example 2), polarization increases with binding, while at the same time the F1-P1 fluorescence is quenched.

1. Homogeneous assay method of quantitative measurement of kinase, phosphatase and phosphodiesterase (PDE) reactions, characterized in that the kinase, phosphatase or phosphodiesterase is allowed to react with a fluorescent, phosphorylatable or dephosphorylatable substrate in the presence of a polycationic polymer containing quencher groups and the change in phosphorylation is determined by way of the change in fluorescence.

2. Assay method according to claim 1, in which the polycationic polymer is polyethylenimine, polyarginine, polylysine and/oder polyhistidine.

3. Assay method according to claim 1, in which the quencher is Dabcyl, QSY35 or another dye suitable for energy transfer, which itself is non-fluorescent.

4. Assay method according to claim 1, in which the fluorescent label is fluorescein, EDANS, rhodamine, Cy5, EvoBlue dyes, coumarins and/or Alexa dyes.

5. Assay method according to claim 1, in which the measurement is carried out kinetically.

6. Assay method according to claim 1, in which the measurement is carried out in parallel/simultaneously in a microtitre plate.

7. Assay method according to claim 1, in which the change in fluorescence is either the change in fluorescence intensity or the change in fluorescence lifetime.

8. Assay method according to claim 1, in which the measurement is used for discovering active compounds which influence the kinase, phosphatase or phosphodiesterase reaction investigated.
